SEQUENCE LISTING (13 sequences)

SEQ ID NO: 1 - protein, 205 aa, Artificial Sequence
  Trap binding to human VEGF-A
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 2 - protein, 107 aa, Artificial Sequence
  VL of 047 Ab-6
DIQMTQSPSSLSASVGDRVTITCSASQDVTTAVAWYQQKPGKAPKLLIYSASFPYRGVPSRFSGSGSGTDFTLTISSLQPEDFATYYCQQYMYHPSTFGQGTKVEIK

SEQ ID NO: 3 - protein, 118 aa, Artificial Sequence
  VH of 047 Ab-6
EVQLVESGGGLVQPGGSLRLSCAASYFPFSDSWIHWVRQAPGKGLEWVAWISPYGGSSYYADSVQDRFTISADTSKNTAYLQMNSLRAEDTAVYYCAKRHWPGGFDHWGQGTLVTVSS

SEQ ID NO: 4 - protein, 443 aa, Artificial Sequence
  H1 heavy chain
EVQLVESGGGLVQPGGSLRLSCAASYFPFSDSWIHWVRQAPGKGLEWVAWISPYGGSSYYADSVQDRFTISADTSKNTAYLQMNSLRAEDTAVYYCAKRHWPGGFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 5 - protein, 666 aa, Artificial Sequence
  H2 heavy chain
QVQLVQSGAEVKKPGASVKVSCKASGYTFTNYVMHWVRQAPGQRLEWMGYINPYNDGTKYSEKFKGRVTITRDTSASTAYMELSSLRSEDTAVYYCARGGLFTFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 666 aa, Artificial Sequence
  H3 heavy chain
QVQLVQSGAEVKKPGASVKVSCKASGYTFTNYVMHWVRQAPGQRLEWMGYINPYNDGTKYSEKFKGRVTITRDTSASTAYMELSSLRSEDTAVYYCARGGLFTFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 7 - protein, 563 aa, Artificial Sequence
  H4 heavy chain
QVQLVQSGAEVKKPGASVKVSCKASGYTFTNYVMHWVRQAPGQRLEWMGYINPYNDGTKYSEKFKGRVTITRDTSASTAYMELSSLRSEDTAVYYCARGGLFTFDNWGQGTLVTVSSGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 439 aa, Artificial Sequence
  L1 light chain
DIVMTQSPLSLPVTPGEPASISCRSSQSLVHSKGNTYLHWYLQKPGQSPQLLIYRVSKRFSGVPDRFSGSGSGTDFTLKISRVEAGDVGVYYCSQSTHVPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 9 - protein, 219 aa, Artificial Sequence
  L2 light chain
DIVMTQSPLSLPVTPGEPASISCRSSQSLVHSKGNTYLHWYLQKPGQSPQLLIYRVSKRFSGVPDRFSGSGSGTDFTLKISRVEAGDVGVYYCSQSTHVPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 558 aa, Artificial Sequence
  L3 light chain
DIVMTQSPLSLPVTPGEPASISCRSSQSLVHSKGNTYLHWYLQKPGQSPQLLIYRVSKRFSGVPDRFSGSGSGTDFTLKISRVEAGDVGVYYCSQSTHVPFTFGQGTKVEIKGGGGSGGGGSGGGGSSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 234 aa, Artificial Sequence
  Human PD-L1-His
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTSGGGGSHHHHHHH

SEQ ID NO: 12 - protein, 354 aa, Artificial Sequence
  Human VEGF-A-mFc
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRSGGGGSVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 134 aa, Artificial Sequence
  Human VEGF-A-His
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRSGGGGSHHHHHHH

(19) United States
(12) Patent Application Publication
(10) Pub. No.: US 2022/0073620 A1
(43) Pub. Date: Mar. 10, 2022

(54) ANTIBODY FUSION PROTEIN, PREPARATION METHOD THEREFOR AND APPLICATION THEREOF

(71) Applicant: CHANGCHUN GENESCIENCE PHARMACEUTICAL CO., LTD., Changchun, Jilin (CN)

(72) Inventors: Xiao FENG, Changchun, Jilin (CN); Tao WANG, Changchun, Jilin (CN); Lei JIN, Changchun, Jilin (CN); Hongrui GUO, Changchun, Jilin (CN); Shuang LIU, Changchun, Jilin (CN); Ning HAN, Changchun, Jilin (CN); Yangqiu LIANG, Changchun, Jilin (CN); Yuheng CHEN, Changchun, Jilin (CN)

(73) Assignee: CHANGCHUN GENESCIENCE PHARMACEUTICAL CO., LTD., Changchun, Jilin (CN)

(21) Appl. No.: 17/418,838

(22) PCT Filed: Dec. 26, 2019
(86) PCT No.: PCT/CN2019/128585

§ 371 Date: Jun. 27, 2021

(30) Foreign Application Priority Data
Dec. 28, 2018 (CN) 201811620872.2

(51) Int. Cl. C07K 16/28; C07K 16/22; C12N 15/62; A61K 47/68; A61P 35/02

(52) U.S. Cl. CPC C07K 16/2827; C07K 16/22; A61P 35/02; A61K 47/6817; C12N 15/62

(57) ABSTRACT

Provided are an antibody fusion protein, a preparation method thereof and an application thereof. The antibody fusion protein is high in expression quantity, and the transient expression quantity in mammalian cells 293E is 100-150 mg/L; the antibody fusion protein is high in assembly rate, and the correct assembly rate exceeds 95%; the antibody fusion protein has a high affinity, and a single-sided antibody/fusion protein and antigen binding KD value is equivalent to a positive control monoclonal antibody/fusion protein and antigen binding KD value; the antibody fusion protein is convenient to purify, and the purity can reach more than 95% in one-step purification by using Protein A or Protein L, and the tumor inhibition rate in a pharmacodynamic experiment animal can reach up to 92%.

### CROSS REFERENCE TO RELATED APPLICATIONS

This application claims the priority of Chinese Patent Application No. 201811620872.2, filed to China National Intellectual Property Administration on Dec. 28, 2018, and titled with “ANTIBODY FUSION PROTEIN, PREPARATION METHOD THEREFOR AND APPLICATION THEREOF”, and the disclosures of which are hereby incorporated by reference.

### FIELD

The present disclosure relates to the field of medicine, specifically to an antibody fusion protein, preparation method thereof and application thereof.

### BACKGROUND

A bispecific monoclonal antibody (BsAb) is a special antibody that is artificially made to bind two different antigens at the same time. Bispecific antibodies can recognize both tumor target cells and immune effector cells, so they have dual functions of antibody specificity and mediating the cytotoxicity of effector cells. Bispecific antibodies can recruit effector cells at tumor sites and activate effector cells to exert anti-tumor effects. The mechanism of killing tumor cells mediated by bispecific antibodies includes cell proliferation, cytokine release, cytotoxic peptides and regulation of enzymes. In vivo and clinical studies have proved that bispecific antibody-mediated immunotherapy can treat tumors in some animals, and clinically can mitigate the condition of patients with tumor and prolong their life. Therefore, the application of bispecific antibody-mediated immunocompetent cells in tumor therapy has a good prospect.

Bispecific antibodies are not nature products and can only be prepared artificially. Bi- or multi-specific antibodies in the art can bind to at least two antigens and can be produced by cell fusion, chemical conjugation or recombinant DNA technology. Recently, a wide variety of recombinant bispecific antibody structures have been developed, such as tetravalent bispecific antibodies by fusion of, for example, an IgG antibody and a single-chain domain (Coloma, M. J., et al., Nature Biotech. 15 (1997) 159-163; WO2001077342; and Morrison, S. L., Nature Biotech. 25 (2007) 1233-1234). In addition, many other new forms that can bind to more than two antigens have been developed, in which the main structure of the antibody (IgA, IgD, IgE, IgG or IgM) is no longer limited to, such as diabodies, triabodies or tetrabodies, minibodies and several single-chain forms (scFv, Bis-scFv) (Holliger, P, et al., Nature Biotech. 23 (2005) 1126-1136; Fischer, N., and Léger, O., Pathobiology 74 (2007) 3-14; Shen, J., et al., Journal of Immunogical Methods 318 (2007) 65-74; Wu, C., et al., Nature Biotech. 25 (2007) 1290-1297).

In one method, the cell quadroma technology (Milstein, C. and A. C. Cuello, Nature, 305 (1983) 537-40) is utilized to produce a bispecific antibody that is very similar to a natural antibody. The cell quadroma technology is based on the somatic fusion of two different hybridoma cell lines expressing murine monoclonal antibodies with the desired bispecific antibody specificity. Because of the random pairing of two different heavy and light chains of antibodies in the hybridoma cell lines, up to 10 different antibody types will be generated, of which only one is the desired functional bispecific antibody. Due to the presence of mismatched by-products and significantly low yields, complicated purification procedures are required (Morrison, S. L., Nature Biotech 25 (2007) 1233-1234). Generally, if recombinant expression technology is used, the same problem of mismatch by-products still exists.

A method used to avoid the problem of mismatch by-products is called “knobs-into-holes”. The purpose is to force the heavy chains from two different antibodies to pair with each other by introducing mutation into the CH3 domain to modify the contact interface. In one chain, amino acids with large volume are replaced by amino acids with short side chains to form a “hole”. Conversely, amino acids with a large side chain are introduced to the other CH3 domain to form a “knob”. By co-expressing these two heavy chains, a higher yield of heterodimer form (“knob-hole”) compared with homodimer form (“hole-hole” or “knob-knob”) was observed (Ridgway, J. B., Presta, L. G., Carter, P. and WO 1996027011). The percentage of heterodimer can be further increased by reconstruction of the interaction interface of the two CH3 domains using phage display method and introduction of disulfide bonds to stabilize the heterodimer (Merchant, A. M., et al., Nature Biotech 16 (1998) 677-681; Atwell, S., Ridgway, J. B., Wells, J. A., Carter, P, J. Mol. Biol. 270 (1997) 26-35). An important constraint of this strategy is that the light chains of the two parent antibodies must be the same to prevent mismatches and formation of inactive molecules.

In addition to the “knob-hole” structure, the Fc pairing of different half-antibodies can also be achieved through the strand-exchange engineered domain (SEED) technology of IgG and IgA CH3 (Davis, J. H., et al., Protein Eng. Des. Sel., 2010, 23(4): 195-202).

In order to solve the problem of the incorrect assembly of different light chains, a new process of double-cell line expressing half-antibodies separately and in vitro assembly has been developed recently. Inspired by the half-antibody random exchange process of human IgG4 antibodies naturally occurring under physiological conditions, GenMab has developed FAE (Fab-arm exchange) bifunctional antibody technology (Gramer, M. J., et al., MAbs 2013, 5(6): 962-973). Introducing two point mutations, K409R and F405L, into the IgG1 heavy chain CH3 domains of the two target antibodies respectively, can produce half-antibody exchange rearrangement similar to that of IgG4 antibodies. Two different IgG1 antibodies after mutation were expressed in two CHO cell lines respectively, and the assembly between the light and heavy chains of each half-antibody was completed. After protein A affinity purification, precise assembly between heterogeneous half-antibodies can be achieved in vitro by using a mild oxidant system.

In addition to sharing light chains with the same sequence or performing in vitro assembly, the correct assembly of light chains of antibodies can also be facilitated by Crossmab technology. A representative product is Roche's Ang-2/VEGF CrossMab CH1-CL. Based on the modification of “knobs-into-holes”, Crossmab technology exchanged CL and CH1 in the Fab domain of Ang-2 antibody and remained the Fab domain of VEGF antibody unchanged. The light chain of the modified Ang-2 antibody is not easily mismatched with the heavy chain of the VEGF antibody, and the “knob-hole” structure can promote the heterodimerization of the two heavy chains (Schaefer, W, et al., Proc Natl. Acad. Sci. USA, 2011, 108(27): 11187-11192).

Moreover, two single-chain antibodies (scFv) or two Fabs can be linked through a peptide to form a bifunctional antibody fragment. A representative product is BiTE (bispecific T-cell engager) series products developed by Micromet in German. This series of products is generated by linking anti-CD3 single-chain antibodies with the single-chain antibodies against different anti-tumor cell surface antigens through a peptide (Baeuerle, P A., et al., Cancer Res., 2009, 69(12): 4941-4944). The advantage of such antibody structure is that it has a small molecular weight, can be expressed in prokaryotic cells, and does not require the consideration of incorrect assembly; while the disadvantage is that it cannot mediate some corresponding biological functions due to a lack of antibody Fc fragment, and its half-life is short.

In addition, the related bispecific antibody proteins in the prior art also have the disadvantages of low expression levels in transient transfection and low affinity, and the complexity of some purification processes, which makes it difficult to meet the needs of industrial production.

### SUMMARY

In view of the above, the present disclosure provides an antibody fusion protein, preparation method thereof and application thereof. The bispecific antibody fusion protein has advantages of high expression level, high assembly rate, high affinity, and easiness of purification. The purity of one-step purification using Protein A or Protein L can reach more than 95%.

In order to achieve the above objects of the present disclosure, the present disclosure provides the following technical solutions.

The present disclosure provides an antibody fusion protein, comprising

(I) an antibody that specifically binds to a first antigen,

(II) a flexible peptide, and

(III) a fusion protein that specifically binds to a second antigen.

In some specific embodiments of the present disclosure, the antibody comprises one or more fragments selected from light chain variable region (VL), light chain constant region (CL), heavy chain variable region (VH), heavy chain constant region 1 (CH1), heavy chain constant region 2 (CH2), and heavy chain constant region 3 (CH3).

In some specific embodiments of the present disclosure, the antibody further comprises a hinge region.

In some specific embodiments of the present disclosure, the antibody fusion protein comprises

a1) light chain variable region and light chain constant region of the antibody that specifically binds to the first antigen, the flexible peptide and the fusion protein that specifically binds to the second antigen, represented as VL-CL-Linker-Trap, and

b1) heavy chain variable region, heavy chain constant region 1 and partial hinge region of the antibody that specifically binds to the first antigen, the flexible peptide, and the fusion protein that specifically binds to the second antigen, represented as VH-CH1-Partial hinge-Linker-Trap;

or comprises

a2) light chain of the antibody that specifically binds to the first antigen, and

b2) heavy chain variable region and heavy chain constant region 1 of the antibody that specifically binds to the first antigen, the flexible peptide, the fusion protein that specifically binds to the second antigen, heavy chain constant region 2 and heavy chain constant region 3, represented as VH-CH1-Linker-Trap-CH2-CH3;

or comprises

a3) light chain of the antibody that specifically binds to the first antigen, and

b3) heavy chain variable region, heavy chain constant region 1, heavy chain constant region 2 and heavy chain constant region 3 of the antibody that specifically binds to the first antigen, the flexible peptide, and the fusion protein that specifically binds to the second antigen, represented as VH-CH1-CH2-CH3-Linker-Trap;

or comprises

a4) light chain variable region of the antibody that specifically binds to the first antigen, the flexible peptide, the fusion protein that specifically binds to the second antigen, heavy chain constant region 2 and heavy chain constant region 3, represented as VL-Linker-Trap-CH2-CH3, and

b4) heavy chain variable region of the antibody that specifically binds to the first antigen, the flexible peptide, the fusion protein that specifically binds to the second antigen, heavy chain constant region 2 and heavy chain constant region 3, represented as VH-Linker-Trap-CH2-CH3.

In some specific embodiments of the present disclosure, the flexible peptide comprises a sequence of (G4S)ₙ, wherein n is an integer greater than 0; preferably an integer of 1-10.

In some specific embodiments of the present disclosure, the light chain constant region (CL) and the heavy chain constant region 1 (CH1) form a heterodimer, and the terminal cysteine residue in the light chain constant region (CL) and the cysteine residue in the hinge region of heavy chain form a disulfide bond.

In some embodiments of the present disclosure, the cysteine residues in the hinge regions of heavy chains form a disulfide bond.

In some specific embodiments of the present disclosure, the domain of the heavy chain constant region 3 (CH3) of first heavy chain and the domain of the heavy chain constant region 3 (CH3) of second heavy chain are modified to a structure that facilitates the formation of the antibody fusion protein.

In some specific embodiments of the present disclosure, the modification comprises

c) modification to the domain of the heavy chain constant region 3 (CH3) of the first heavy chain: in the interface between the domain of the heavy chain constant region 3 (CH3) of the first heavy chain and the domain of the heavy chain constant region 3 (CH3) of the second heavy chain of bivalent bispecific antibody, an amino acid residue in the domain of the heavy chain constant region 3 (CH3) of the first heavy chain is replaced with an amino acid residue with a volume larger than the original amino acid residue to form a knob in the domain of the heavy chain constant region 3 (CH3) of the first heavy chain, wherein the knob is capable of inserting into a hole of the domain of the heavy chain constant region 3 (CH3) of the second heavy chain, and

d) modification to the domain of the heavy chain constant region 3 (CH3) of the second heavy chain: in the interface between the domain of the heavy chain constant region 3 (CH3) of the second heavy chain and the domain of the heavy chain constant region 3 (CH3) of the first heavy chain of bivalent bispecific antibody, an amino acid residue in the domain of the heavy chain constant region 3 (CH3) of the second heavy chain is replaced with an amino acid residue with a volume smaller than the original amino acid residue to form a hole in the domain of the heavy chain constant region 3 (CH3) of the second heavy chain, wherein the hole is capable of holding the knob of the domain of the heavy chain constant region 3 (CH3) of the first heavy chain.

In some specific embodiments of the present disclosure, wherein in the heavy chain,

The amino acid residue with a volume larger than the original amino acid residue is selected from the group consisting of arginine, phenylalanine, tyrosine, and tryptophan; and

The amino acid residue with a volume smaller than the original amino acid residue is selected from the group consisting of alanine, serine, threonine, and valine.

In some specific embodiments of the present disclosure,

(IV) the heavy chain with an amino acid sequence as shown in SEQ ID NO: 4, SEQ ID NO: 5, SEQ ID NO: 6 or SEQ ID NO: 7, and

(V) the light chain with an amino acid sequence as shown in SEQ ID NO: 8, SEQ ID NO: 9 or SEQ ID NO: 10;

or (VI) an amino acid sequence derived from the amino acid sequence described in (IV) or (V) by substitution, deletion or addition of one or more amino acids, and functionally identical or similar to the amino acid sequence described in (IV) or (V);

or (VII) an amino acid sequence with more than 90% homology with the sequence described in (IV) or (V);

or (VIII) an amino acid sequence that has the same functional fragment or functional variant as the sequence described in (IV) or (V);

wherein the antibody fusion protein specifically binds to hPD-L1 and hVEGF-A; and

The first antigen is hPD-L1 and the second antigen is hVEGF-A.

In some specific embodiments of the present disclosure, wherein the one or more amino acids is 2-10 amino acids.

In some specific embodiments of the present disclosure, the antibody fusion protein comprises

(IX) a heavy chain with an amino acid sequence of SEQ ID NO: 4, and a light chain with an amino acid sequence of SEQ ID NO: 8; or

(X) a heavy chain with an amino acid sequence of SEQ ID NO: 5, and a light chain with an amino acid sequence of SEQ ID NO: 9; or

(XI) a heavy chain with an amino acid sequence of SEQ ID NO: 6, and a light chain with an amino acid sequence of SEQ ID NO: 9; or

(XII) a heavy chain with an amino acid sequence of SEQ ID NO: 7, and a light chain with an amino acid sequence of SEQ ID NO: 10.

In some specific embodiments of the present disclosure, the antibody fusion protein specifically binds to hPD-L1 and hVEGF-A, wherein the first antigen is hPD-L1, and the second antigen is hVEGF-A.

In some specific embodiments of the present disclosure, the antibody fusion protein with FabT structure has an amino acid sequence of heavy chain as shown in SEQ ID NO: 4, and an amino acid sequence of light chain as shown in SEQ ID NO: 8; the antibody fusion protein with FTF structure has an amino acid sequence of heavy chain as shown in SEQ ID NO: 5, and an amino acid sequence of light chain as shown in SEQ ID NO: 9; the antibody fusion protein with IgGT structure has an amino acid sequence of heavy chain as shown in SEQ ID NO: 6, and an amino acid sequence of light chain as shown in SEQ ID NO: 9; and the antibody fusion protein with FvT structure has an amino acid sequence of heavy chain as shown in SEQ ID NO: 7, and an amino acid sequence of light chain as shown in SEQ ID NO: 10.

The present disclosure also provides a nucleic acid molecule encoding the antibody fusion protein, comprising

(XIII) a nucleic acid encoding the heavy chain variable region as shown in SEQ ID NO: 4, SEQ ID NO: 5, SEQ ID NO: 6 or SEQ ID NO: 7, and a nucleic acid encoding the light chain variable region as shown in SEQ ID NO: 8, SEQ ID NO: 9 or SEQ ID NO: 10; or

(XIV) a nucleic acid having complementary sequence of the heavy chain variable region as shown in SEQ ID NO: 4, SEQ ID NO: 5, SEQ ID NO: 6 or SEQ ID NO: 7, and a nucleic acid having complementary sequence of the light chain variable region as shown in SEQ ID NO: 8, SEQ ID NO: 9 or SEQ ID NO: 10; or

(XV) a nucleotide sequence encoding the same protein as the nucleotide sequence described in (XIII) or (XIV), but different from the nucleotide sequence described in (XIII) or (XIV) due to the degeneracy of genetic code; or

(XVI) a sequence with more than 90% homology with the sequence described in (XIII) or (XIV) or (XV).

In some specific embodiments of the present disclosure, the nucleic acid molecule comprises a nucleotide sequence derived from the nucleotide sequence described in (XIII) or (XIV) or (XV) or (XVI) by substitution, deletion or addition of one or more nucleotide, and functionally identical or similar to the nucleotide sequence described in (XIII) or (XIV) or (XV) or (XVI), wherein the one or more amino acids is 2-10 amino acids.

The present disclosure also provides an expression vector, comprising the nucleic acid molecule and a cell transformed with the expression vector.

The present disclosure also provides a complex comprising the antibody fusion protein covalently linked to an isotope, an immunotoxin and/or a chemical drug.

The present disclosure also provides a conjugate, formed by coupling the antibody fusion protein and/or the complex with a solid medium or a semi-solid medium.

The present disclosure also provides use of the antibody fusion protein and/or the complex and/or the conjugate for the manufacture of a medicament for the treatment of a disease and/or a composition for the diagnose of a disease; wherein the disease is selected from the group consisting of breast cancer, lung cancer, gastric cancer, intestinal cancer, esophageal cancer, ovarian cancer, cervical cancer, kidney cancer, bladder cancer, pancreatic cancer, glioma, and melanoma.

The present disclosure also provides a pharmaceutical composition comprising the antibody fusion protein and/or the complex and/or the conjugate.

The present disclosure also provides a kit comprising the antibody fusion protein and/or the complex and/or the conjugate.

The present disclosure also provides a method for treating a disease with the antibody fusion protein and/or the complex and/or the conjugate, wherein the disease is selected from the group consisting of breast cancer, lung cancer, gastric cancer, intestinal cancer, esophageal cancer, ovarian cancer, cervical cancer, kidney cancer, bladder cancer, pancreatic cancer, non-Hodgkin's lymphoma, chronic lymphoma leukemia, multiple myeloma, acute myeloid leukemia, acute lymphoma leukemia, glioma, melanoma, diabetic macular edema, and wet macular degeneration.

The present disclosure also provides a method for producing the antibody fusion protein, comprising transforming a host cell with the expression vector, culturing the host cell under conditions that allow the synthesis of the antibody fusion protein, and recovering the antibody fusion protein from the culture.

The antibody fusion proteins provided by the present disclosure have a high expression level with transient expression of 100-150 mg/L in mammalian cell 293E; a high assembly rate with a correct assembly rate of more than 95%; a high affinity with a binding KD value of single-sided antibody/fusion protein to the antigen comparable to that of the positive control monoclonal antibody/fusion protein to the antigen; and easiness of purification with a purity of one-step purification using Protein A or Protein L up to more than 95%.

### BRIEF DESCRIPTION OF DRAWINGS

In order to illustrate examples of the present disclosure or technical solutions in the prior art more clearly, drawings required to be used in the description of the examples or prior art will be introduced briefly below.

FIG. 1 shows a schematic diagram of the bispecific antibody fusion protein with FabT structure.

FIG. 2 shows a schematic diagram of the bispecific antibody fusion protein with FTF structure.

FIG. 3 shows a schematic diagram of the bispecific antibody fusion protein with IgGT structure.

FIG. 4 shows a schematic diagram of the bispecific antibody fusion protein with FvT structure.

FIG. 5 shows the SDS-PAGE results of the transient expression of bispecific antibody fusion proteins with FabT, FTF, IgGT and FvT structures. M indicates Maker; Lanes 1 and 2 represent the non-reducing electrophoresis and reducing electrophoresis of FabT, respectively; Lanes 3 and 4 represent the non-reducing electrophoresis and reducing electrophoresis of FTF, respectively; Lanes 5 and 6 represent the non-reducing electrophoresis and reducing electrophoresis of IgGT, respectively; and Lanes 7 and 8 represent the non-reducing electrophoresis and reducing electrophoresis of FvT, respectively.

FIG. 6A and FIG. 6B show the ELISA results of the supernatants from transient expression of bispecific antibody fusion proteins with FabT, FTF, IgGT and FvT structures.

FIG. 7 shows the SDS-PAGE results of bispecific antibody fusion proteins with FabT, FTF, IgGT and FvT structures after purification. M indicates Marker; Lanes 1 and 2 represent the non-reducing electrophoresis of FabT purified by Protein L; Lanes 2 and 4 represent the reducing electrophoresis of FTF purified by Mabselect Sure; Lanes 5 and 6 represent the non-reducing electrophoresis of IgGT purified by Mabselect Sure; and Lanes 7 and 8 represent the reducing electrophoresis of FvT purified by Mabselect Sure.

FIG. 8 shows the ELISA results of bispecific antibody fusion proteins with FabT, FTF, IgGT and FvT structures after purification.

FIG. 9 shows the detection results of the activity of bispecific antibody fusion proteins with FabT, FTF, IgGT and FvT structures blocking the cellular VEGFA signaling pathway.

FIG. 10 shows the detection results of T cell activation activity of bispecific antibody fusion proteins with FabT, FTF, IgGT and FvT structures.

FIG. 11 shows the results of animal pharmacodynamic experiments of bispecific antibody fusion proteins with FabT, FTF, IgGT and FvT structures.

### DETAILED DESCRIPTION

The present disclosure discloses an antibody fusion protein, a preparation method thereof and application thereof. In view of the content herein, those skilled in the art can make appropriate modifications to the process parameters. It should be particularly indicated that, all similar replacements and changes are obvious for those skilled in the art, which are deemed to be included in the present disclosure. The methods and uses of the present disclosure have been described by way of preferred embodiments, and it will be apparent to those skilled in the art that changes as well as appropriate modifications and combinations of the methods and uses described herein may be made without departing from the content, spirit and scope of the present disclosure, to achieve and apply the techniques of the present disclosure.

The bispecific antibody fusion protein of the present disclosure comprises

a). light chain variable region and light chain constant region of the antibody that specifically binds to the first antigen, the flexible peptide and the fusion protein that specifically binds to the second antigen, represented as VL-CL-Linker-Trap, and

b). heavy chain variable region, heavy chain constant region 1 and partial hinge region of the antibody that specifically binds to the first antigen, the flexible peptide, and the fusion protein that specifically binds to the second antigen, represented as VH-CH1-Partial hinge-Linker-Trap; or

c). light chain of the antibody that specifically binds to the first antigen, and

d). heavy chain variable region and heavy chain constant region 1 of the antibody that specifically binds to the first antigen, the flexible peptide, the fusion protein that specifically binds to the second antigen, heavy chain constant region 2 and heavy chain constant region 3, represented as VH-CH1-Linker-Trap-CH2-CH3; or

e). light chain of the antibody that specifically binds to the first antigen, and

f). heavy chain variable region, heavy chain constant region 1, heavy chain constant region 2 and heavy chain constant region 3 of the antibody that specifically binds to the first antigen, the flexible peptide, and the fusion protein that specifically binds to the second antigen, represented as VH-CH1-CH2-CH3-Linker-Trap; or

g). light chain variable region of the antibody that specifically binds to the first antigen, the flexible peptide, the fusion protein that specifically binds to the second antigen, heavy chain constant region 2 and heavy chain constant region 3, represented as VL-Linker-Trap-CH2-CH3, and

h). heavy chain variable region of the antibody that specifically binds to the first antigen, the flexible peptide, the fusion protein that specifically binds to the second antigen, heavy chain constant region 2 and heavy chain constant region 3, represented as VH-Linker-Trap-CH2-CH3.

Further, the flexible peptide comprises a sequence of (G4S)ₙ, wherein n is an integer greater than 0.

Further, CL and CH1 form a heterodimer, and the terminal cysteine residue in CL and the cysteine residue in the hinge region of heavy chain form a disulfide bond; or

further, the cysteine residues in the hinge regions of heavy chains form a disulfide bond.

Further, the CH3 domain of first heavy chain and the CH3 domain of second heavy chain are modified to a structure that facilitates the formation of the antibody fusion protein.

The bispecific antibody fusion protein can be modified, and the modification comprises

a) modification to the CH3 domain of the first heavy chain: in the interface between the CH3 domain of the first heavy chain and the CH3 domain of the second heavy chain of bivalent bispecific antibody, an amino acid residue in the CH3 domain of the first heavy chain is replaced with an amino acid residue with a volume larger than the original amino acid residue to form a knob in the CH3 domain of the first heavy chain, wherein the knob is capable of inserting into a hole of the CH3 domain of the second heavy chain, and

b) modification to the CH3 domain of the second heavy chain: in the interface between the CH3 domain of the second heavy chain and the CH3 domain of the first heavy chain of bivalent bispecific antibody, an amino acid residue in the CH3 domain of the second heavy chain is replaced with an amino acid residue with a volume smaller than the original amino acid residue to form a hole in the CH3 domain of the second heavy chain, wherein the hole is capable of holding the knob of the CH3 domain of the first heavy chain.

The amino acid residues with a volume larger than the original amino acid residue is selected from the group consisting of arginine, phenylalanine, tyrosine, and tryptophan.

The amino acid residues with a volume smaller than the original amino acid residue is selected from the group consisting of alanine, serine, threonine, and valine.

Further, the bispecific antibody fusion protein is a bispecific antibody that specifically binds to hPD-L1 and hVEGF-A. The bispecific antibody fusion protein with FabT structure has an amino acid sequence of heavy chain as shown in SEQ ID NO: 4, and an amino acid sequence of light chain as shown in SEQ ID NO: 8; the bispecific antibody fusion protein with FTF structure has an amino acid sequence of heavy chain as shown in SEQ ID NO: 5, and an amino acid sequence of light chain as shown in SEQ ID NO: 9; the bispecific antibody fusion protein with IgGT structure has an amino acid sequence of heavy chain as shown in SEQ ID NO: 6, and an amino acid sequence of light chain as shown in SEQ ID NO: 9; and the bispecific antibody fusion protein with FvT structure has an amino acid sequence of heavy chain as shown in SEQ ID NO: 7, and an amino acid sequence of light chain as shown in SEQ ID NO: 10.

The method for producing the bispecific antibody fusion proteins of the present disclosure comprises the following steps:

a) transforming a host cell with

a vector comprising a nucleic acid molecule encoding light chain variable region and light chain constant region of the antibody that specifically binds to the first antigen, the flexible peptide and the fusion protein that specifically binds to the second antigen, and

a vector comprising a nucleic acid molecule encoding heavy chain variable region, heavy chain constant region 1 and partial hinge region of the antibody that specifically binds to the first antigen, the flexible peptide, and the fusion protein that specifically binds to the second antigen; or

a vector comprising a nucleic acid molecule encoding light chain of the antibody that specifically binds to the first antigen, and

a vector comprising a nucleic acid molecule encoding heavy chain variable region and heavy chain constant region 1 of the antibody that specifically binds to the first antigen, the flexible peptide, the fusion protein that specifically binds to the second antigen, heavy chain constant region 2 and heavy chain constant region 3; or

a vector comprising a nucleic acid molecule encoding light chain of the antibody that specifically binds to the first antigen, and

a vector comprising a nucleic acid molecule encoding heavy chain variable region, heavy chain constant region 1, heavy chain constant region 2 and heavy chain constant region 3 of the antibody that specifically binds to the first antigen, the flexible peptide, and the fusion protein that specifically binds to the second antigen; or

a vector comprising a nucleic acid molecule encoding light chain variable region of the antibody that specifically binds to the first antigen, the flexible peptide, the fusion protein that specifically binds to the second antigen, heavy chain constant region 2 and heavy chain constant region 3, and

a vector comprising a nucleic acid molecule encoding heavy chain variable region of the antibody that specifically binds to the first antigen, the flexible peptide, the fusion protein that specifically binds to the second antigen, heavy chain constant region 2 and heavy chain constant region 3;

b) culturing the host cell under conditions that allow the synthesis of the bispecific antibody fusion protein; and

c) recovering the antibody fusion protein from the culture.

The host cell of the present disclosure comprises

a vector comprising a nucleic acid molecule encoding light chain variable region and light chain constant region of the antibody that specifically binds to the first antigen, the flexible peptide and the fusion protein that specifically binds to the second antigen, and

a vector comprising a nucleic acid molecule encoding heavy chain variable region, heavy chain constant region 1 and partial hinge region of the antibody that specifically binds to the first antigen, the flexible peptide, and the fusion protein that specifically binds to the second antigen; or

a vector comprising a nucleic acid molecule encoding light chain of the antibody that specifically binds to the first antigen, and

a vector comprising a nucleic acid molecule encoding heavy chain variable region and heavy chain constant region 1 of the antibody that specifically binds to the first antigen, the flexible peptide, the fusion protein that specifically binds to the second antigen, heavy chain constant region 2 and heavy chain constant region 3; or

a vector comprising a nucleic acid molecule encoding light chain of the antibody that specifically binds to the first antigen, and

a vector comprising a nucleic acid molecule encoding heavy chain variable region, heavy chain constant region 1, heavy chain constant region 2 and heavy chain constant region 3 of the antibody that specifically binds to the first antigen, the flexible peptide, and the fusion protein that specifically binds to the second antigen; or

a vector comprising a nucleic acid molecule encoding light chain variable region of the antibody that specifically binds to the first antigen, the flexible peptide, the fusion protein that specifically binds to the second antigen, heavy chain constant region 2 and heavy chain constant region 3, and

a vector comprising a nucleic acid molecule encoding heavy chain variable region of the antibody that specifically binds to the first antigen, the flexible peptide, the fusion protein that specifically binds to the second antigen, heavy chain constant region 2 and heavy chain constant region 3.

The composition of the bispecific antibody fusion protein of the present disclosure comprises a therapeutically effective amount of any of the above bispecific antibody fusion proteins and a pharmaceutically acceptable carrier, pharmaceutically acceptable auxiliary or pharmaceutically acceptable excipient; preferably, the composition is a pharmaceutical composition (i.e., a drug) or a diagnostic composition.

Further, the pharmaceutical composition comprises any of the above bispecific antibody fusion proteins and at least one pharmaceutically acceptable excipient.

The bispecific antibody fusion protein of the present disclosure has the following excellent technical effects:

1. high expression level, the transient expression level in mammalian cells 293E is 100-150 mg/L;

2. high assembly rate, the correct assembly rate exceeds 95%;

3. high affinity, the binding KD value of single-sided antibody/fusion protein to the antigen is comparable to that of the positive control monoclonal antibody/fusion protein to the antigen;

4. simple purification process, the purity of one-step purification using Protein A or Protein L can reach more than 95%.

Raw materials, auxiliary materials and reagents used for the antibody fusion proteins, preparation method thereof and application thereof provided by the present disclosure are all purchased from the market.

The present disclosure will be further illustrated by the following examples:

### Example 1 Preparation of Bispecific Antibody Fusion Proteins

### 1. Construction of Transient Transfection Expression Vector for Bispecific Antibody Fusion Proteins Materials

The sequence of Trap (SEQ ID NO: 1) binding to human VEGF-A was derived from Regeneron's listed drug “Eylea” (for the sequence, reference could be made to the sequence listing <210>6 of Chinese patent CN103349781B). The anti-human PD-L1 humanized monoclonal antibody was derived from 047 Ab-6 having the sequence of VL (SEQ ID NO: 2) and the sequence of VH (SEQ ID NO: 3), which was obtained by panning of the natural human source library by Genescience. Reference could be also made to Chinese patent CN201810044303.1 for the coding nucleotides of heavy chain constant region CH1, hinge region and Fc of IgG1, and nucleotides of Kappa chain constant region.

### Methods

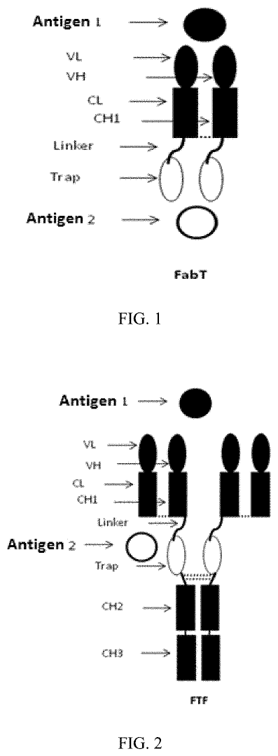

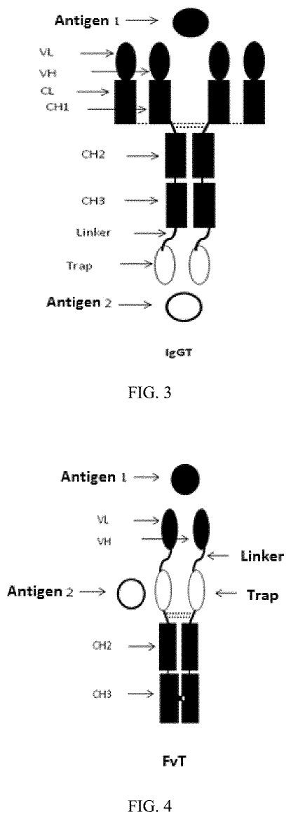

pGS003 was selected to construct the expression vectors for the heavy chain and light chain of bispecific antibody fusion proteins (4 proteins, of which the structure diagrams are shown in FIG. 1 to FIG. 4). Primers were designed according to the coding nucleotides of VEGFR1 domain 2 and VEGFR2 domain 3, the coding nucleotides of VL and VH derived from the anti-human PD-L1 humanized monoclonal antibody 047 Ab-6, the coding nucleotides of heavy chain constant region CH1, hinge region and Fc of IgG1, and nucleotide sequence of Kappa chain constant region, and multiple cloning sites in the vector. After PCR amplification, four heavy chain coding sequences and three light chain coding sequences were cloned into pGS003 by in vitro recombination method (Nanjing GenScript, CloneEZ PCR Cloning Kit), as shown in Table 1. After sequencing to identify the correct insertion of the target gene, the recombinant expression vectors were transformed into E. coli TOP10F′. Then a single colony was picked and inoculated in LB medium containing 100 μg/mL of ampicillin, and cultured with shaking at 37° C. for 16 hours. The plasmids were extracted using endotoxin-removal, large-scale extraction kit of Zymo Research. The obtained plasmids were dissolved in 1 mL of ultrapure water, and the plasmid concentration and OD260/280 were determined with a spectrophotometer. A plasmid with OD260/280 value between 1.8 and 1.9 is considered a relatively pure plasmid DNA.

TABLE 1
List of transient transfection expression vectors for heavy and light chains

| Vector for Heavy | Heavy Chain Amino | Vector for Light | Light Chain Amino |
|---|---|---|---|
| Chain Expression | Acid Sequence | Chain Expression | Acid Sequence |
| H1 | SEQ ID NO: 4 | L1 | SEQ ID NO: 8 |
| H2 | SEQ ID NO: 5 | L2 | SEQ ID NO: 9 |
| H3 | SEQ ID NO: 6 | L3 | SEQ ID NO: 10 |
| H4 | SEQ ID NO: 7 | | |

### 2. Transfection, Expression and Detection in Mammalian 293E Cells

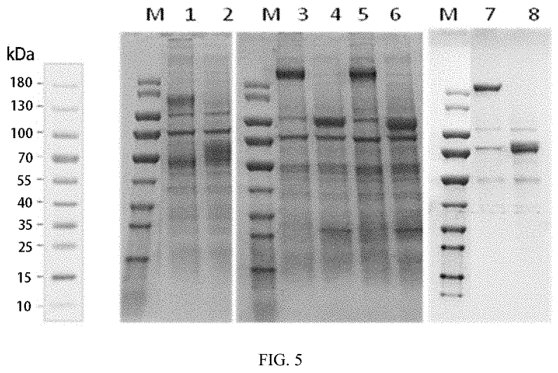

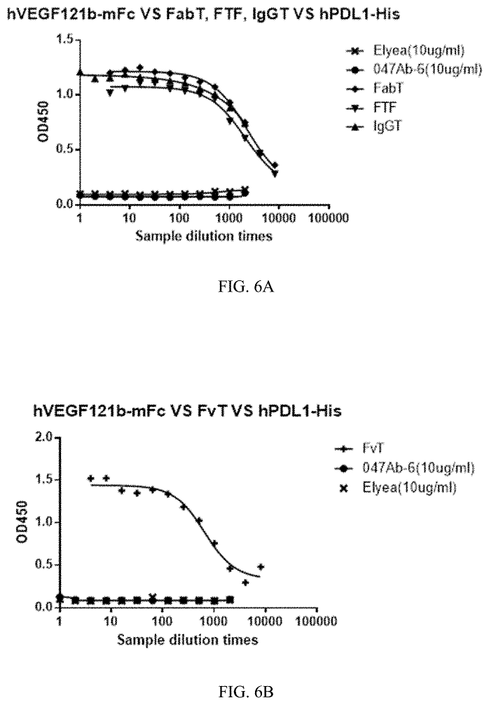

Vectors for the above four heavy chain expression vectors and three light chain expression were constructed. H1 was used to express VH of anti-hPD-L1 and the fusion protein binding hVEGF-A, L1 was used to express VL of anti-hPD-L1 and the fusion protein binding hVEGF-A, H2 was used to express VH of anti-hPD-L1 and the fusion protein binding hVEGF-A, L2 was used to express VL of anti-hPD-L1, H3 was used to express VH of anti-hPD-L1 and the fusion protein binding hVEGF-A, L3 was used to express VL of anti-hPD-L1 and the fusion protein binding hVEGF-A, and H4 was used to express VH of anti-hPD-L1 and the fusion protein binding hVEGF-A. Combinations of the above vectors, H1+L1 (FabT structure), H2+L2 (FTF structure), H3+L2 (IgGT structure), and H4+L3 (FvT structure), were subjected to transient transfection expression in 2 mL 293E system for evaluation, where the linkers in FabT, FTF, IgGT and FvT structures were all (G4S)₃. The expression levels and the ELISA detection value of the antibody binding to human VEGF-A and human PD-L1 were detected. The results are shown in FIG. 5, Table 2, Table 3, FIGS. 6A and 6B. The expression, assembly and binding to antigens of FabT, FTF, IgGT and FvT structures were all fairly good.

293E cells were used to perform amplified transient transfection expression of FabT, FTF, IgGT and FvT structures in Freestyle medium. 24 hours before transfection, 300 mL of 293E cells at 0.5×10⁶ cells/mL were seeded in a 1 L cell culture flask, and cultured in a 37° C., 5% CO₂ incubator with shaking at 120 rpm. During transfection, 300 μL of 293Fectin™ was added to 5.7 mL Opti-MEM™. After mixing well, the mixture was incubated at room temperature for 2 minutes. Meanwhile, 300 μg of the expression plasmids for FabT structure and FTF structure were diluted to 6 mL with OPtiMEM, respectively. The diluted transfection reagent 293 fectin and plasmids were mixed thoroughly and incubated at room temperature for 15 minutes. After that, the mixture was added to cells and mixed well, and cultured in a 37° C., 5% CO₂ incubator with shaking at 120 rpm for 7 days.

TABLE 2
Transient transfection assembly rates of FabT, FTF, IgGT and FvT

| Lane | Band | Peak | Average | Trace | | |
|---|---|---|---|---|---|---|
| Number | Number | Int | Int | Int × mm | Peak × Trace | Band % |
| 1 | 1 | 38.412 | 21.984 | 209.394 | 8043.242 | 99% |
| 1 | 2 | 8.482 | 7.951 | 8.415 | 67.44 | |
| 2 | 1 | 68.568 | 26.215 | 360.673 | 24730.626 | 99% |
| 2 | 2 | 10.163 | 8.161 | 11.517 | 82.94 | |
| 3 | 1 | 66.907 | 28.937 | 377.712 | 25271.577 | 99% |
| 3 | 2 | 12.136 | 8.815 | 12.439 | 150.959 | |
| 4 | 1 | 55.284 | 16.841 | 178.238 | 9553.71 | 99% |

TABLE 3
Transient transfection expression
levels of FabT, FTF, IgGT and FvT

| Antibody structure | FabT | FTF | IgGT | FvT |
|---|---|---|---|---|
| Expression level (mg/L) | 100 | 150 | 150 | 100 |

### Example 2 Purification and Detection of Preferred Antibodies

Purification of Proteins with FabT Structure

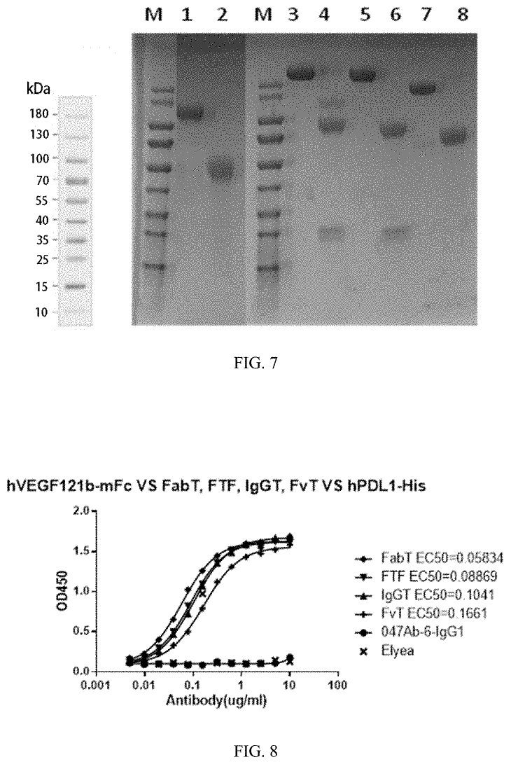

The cell culture medium was centrifuged at 2000 g for 20 min, and the supernatant was collected and then filtered with a 0.22 micron filter membrane. Next, the supernatant was subjected to Protein L (GE) chromatography, the proteins were eluted with 20 mM citrate-sodium citrate, pH 3.0, and then the resultant was adjusted to neutral pH with 1 M Tris base. Purified samples were detected by SDS-PAGE using 4-20% gradient gel (GenScript Biotechnology Co., Ltd.) to detect purified proteins. The results are shown in FIG. 7 and Table 4. The purity of FabT was 95%.

Purification of Proteins with FTF Structure

The cell culture medium was centrifuged at 2000 g for 20 min, and the supernatant was collected and then filtered with a 0.22 micron filter membrane. Next, the supernatant was subjected to Mabselect Sure (GE) chromatography, the proteins were eluted with 20 mM citrate-sodium citrate, pH 3.0, and then the resultant was adjusted to neutral pH with 1 M Tris base. Purified samples were detected by SDS-PAGE using 4-20% gradient gel (GenScript Biotechnology Co., Ltd.) to detect purified proteins. The results are shown in FIG. 7 and Table 4. The purity of FTF was 95%.

Purification of Proteins with IgGT Structure

The cell culture medium was centrifuged at 2000 g for 20 min, and the supernatant was collected and then filtered with a 0.22 micron filter membrane. Next, the supernatant was subjected to Mabselect Sure (GE) chromatography, the proteins were eluted with 20 mM citrate-sodium citrate, pH 3.0, and then the resultant was adjusted to neutral pH with 1 M Tris base. Purified samples were detected by SDS-PAGE using 4-20% gradient gel (GenScript Biotechnology Co., Ltd.) to detect purified proteins. The results are shown in FIG. 7 and Table 4. The purity of IgGT was 95%.

Purification of Proteins with FvT Structure

The cell culture medium was centrifuged at 2000 g for 20 min, and the supernatant was collected and then filtered with a 0.22 micron filter membrane. Next, the supernatant was subjected to Mabselect Sure (GE) chromatography, the proteins were eluted with 20 mM citrate-sodium citrate, pH 3.0, and then the resultant was adjusted to neutral pH with 1 M Tris base. Purified samples were detected by SDS-PAGE using 4-20% gradient gel (GenScript Biotechnology Co., Ltd.) to detect purified proteins. The results are shown in FIG. 7 and Table 4. The purity of FvT was 95%.

TABLE 4
Purities of FabT, FTF, IgGT and FvT after purification

| Lane | Band | Peak | Average | Trace | | |
|---|---|---|---|---|---|---|
| Number | Number | Int | Int | Int × mm | Peak × Trace | Band % |
| 1 | 1 | 50.016 | 21.287 | 202.754 | 10140.944 | 99% |
| 2 | 1 | 67.747 | 27.728 | 264.105 | 17892.321 | 99% |
| 2 | 2 | 2.043 | 1.228 | 0.975 | 1.99 | |
| 3 | 1 | 58.903 | 22.344 | 212.828 | 12536.208 | 99% |
| 3 | 2 | 0.821 | 0.353 | 0.374 | 0.37 | |
| 4 | 1 | 57.728 | 25.974 | 185.551 | 10711.488 | 99% |
| 4 | 2 | 8.191 | 7.931 | 6.295 | 51.562 | |

### Example 3 ELISA Detection of Preferred Antibodies Binding to Human VEGF-A and Human PD-L1

1. Coating the first antigen: Human PD-L1-His (constructed by GeneScience, SEQ ID NO: 11) was diluted with PBS to 1 μg/mL, and then added to a 96-well microtiter plate at 50 μL per well and incubated overnight at 4° C.

2. Blocking: After being washed three times, the plate was blocked with 3% BSA at 250 μL per well, and incubated at 37° C. for 2 hours.

3. Adding candidate antibody: After being washed three times, the candidate antibody was added to the plate, each with 12 samples diluted at a 2-fold concentration gradient with an initial concentration of 10 mg/mL, positive control or negative control was added at 50 μL per well, and incubated at 25° C. for 1 hour.

4. Adding the second antigen: After the plate was washed three times, human VEGF-A-mFc (constructed by GeneScience, SEQ ID NO: 12) was diluted with PBS to 10 μg/mL, and then added to the 96-well microtiter plate at 50 μL per well and incubated at 25° C. for 1 hour.

5. Adding the secondary antibody: After being washed three times, HRP-labeled streptavidin (1:10,000) was added to the plate at 50 per well, and incubated at 25° C. for 1 hour.

6. Color development: After being washed four times, TMB color development solution was added to the plate at 50 μL per well, and developed color shielded from light at room temperature for 10 minutes.

7. Terminating: The stop solution was directly added to the plate at 50 μL per well to terminate the reaction.

8. Detection: After terminating the reaction, the microtiter plate was immediately put into the microplate reader. The OD value at 450 nm was measured, and the original data was saved for sorting. The results are shown in FIG. 8 and Table 5, showing that for the purified FabT, EC₅₀=0.05834; for FTF, EC₅₀=0.08869; for IgGT, EC₅₀=0.1041; and for FvT, EC₅₀=0.1661.

TABLE 5
EC₅₀ of purified FabT, FTF, IgGT and FvT detected by ELISA

| Antibody structure | FabT | FTF | IgGT | FvT |
|---|---|---|---|---|
| EC₅₀ | 0.05834 | 0.08869 | 0.1041 | 0.1661 |

### Example 4 Affinity Determination of Preferred Antibodies

The affinities of FabT and FTF were detected by Biacore T200 instrument. The specific protocols were as follows. Human PD-L1-His (constructed by GeneScience, SEQ ID NO: 11) and human VEGF-A-His (constructed by GeneScience, SEQ ID NO: 13) were coupled to CMS biosensor chip (GE), and then the antibodies of different concentrations were flowed through the chip at a flow rate of 30 μL/min. The binding between the candidate antibody and antigen was performed with a binding time of 120 s and a dissociation time of 300 s. The kinetic fitting was performed using BIAevalution software (GE), and the results of affinity constants were obtained as shown in Table 6 and Table 7. The affinities of FabT, FTF, IgGT and FvT with PD-L1 were 6.16E-10 M, 1.04E-12 M, 6.37E-13 M and 3.37E-09 M, respectively; and the affinities of FabT, FTF, IgGT and FvT with VEGF-A were 1.75E-09 M, 2.00E-09M, 2.77E-08 M and 2.40E-09 M, respectively.

For the bispecific antibodies that specifically bind to hPD-L1 and hVEGF-A in some specific embodiments, the bispecific antibody fusion protein with FabT structure has an amino acid sequence of heavy chain as shown in SEQ ID NO: 4, and an amino acid sequence of light chain as shown in SEQ ID NO: 8; the bispecific antibody fusion protein with FTF structure has an amino acid sequence of heavy chain as shown in SEQ ID NO: 5, and an amino acid sequence of light chain as shown in SEQ ID NO: 9; the bispecific antibody fusion protein with IgGT structure has an amino acid sequence of heavy chain as shown in SEQ ID NO: 6, and an amino acid sequence of light chain as shown in SEQ ID NO: 9; and the bispecific antibody fusion protein with FvT structure has an amino acid sequence of heavy chain as shown in SEQ ID NO: 7, and an amino acid sequence of light chain as shown in SEQ ID NO: 10.

TABLE 6
Results of affinity detection of candidate
bispecific molecules with PD-L1

| Antibody | Ka (1/Ms) | Kd (1/s) | KD (M) | Rmax (RU) |
|---|---|---|---|---|
| FabT | 3.20E+05 | 1.97E−04 | 6.16E−10 | 20.7 |
| FTF | 1.83E+05 | 1.90E−07 | 1.04E−12 | 26.4 |
| IgGT | 2.34E+05 | 1.58E−07 | 6.37E−13 | 33.7 |
| FvT | 1.27E+05 | 4.29E−04 | 3.37E−09 | 18.6 |
| PD-L1 positive | 3.64E+05 | 4.12E−07 | 1.13E−12 | 27.9 |
| antibody | | | | |

TABLE 7
Results of affinity detection of candidate
bispecific molecules with VEGF-A

| Antibody | Ka (1/Ms) | Kd (1/s) | KD (M) | Rmax (RU) |
|---|---|---|---|---|
| FabT | 7.84E+05 | 1.37E−03 | 1.75E−09 | 14.6 |
| FTF | 3.11E+05 | 6.24E−04 | 2.00E−09 | 8.2 |
| IgGT | 3.66E+05 | 1.02E−03 | 2.77E−09 | 9.2 |
| FvT | 3.22E+05 | 7.74E−04 | 2.40E−09 | 8.4 |
| Eylea positive | 6.17E+05 | 9.32E−04 | 1.51E−09 | 13.2 |
| fusion protein | | | | |

### Example 5 Activity Determination of Preferred Antibodies on Blocking Cellular VEGFA Signaling Pathway

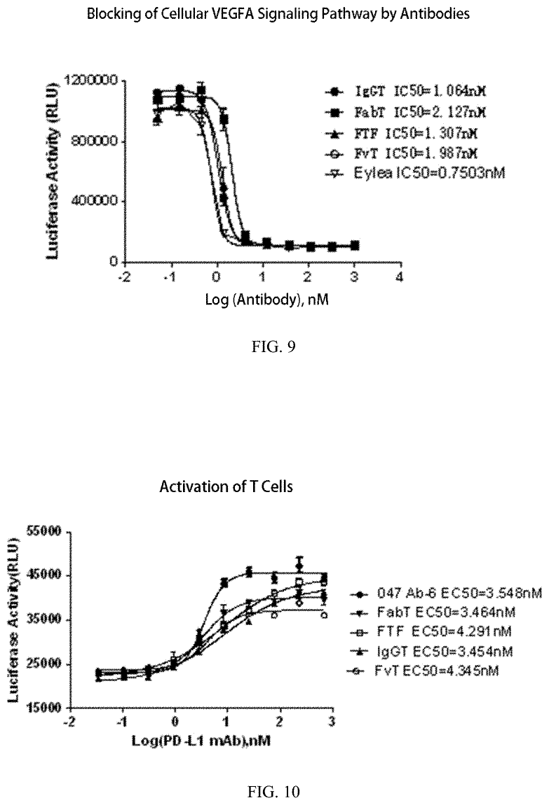

NFAT-RE-Luc2P-KDR-HEK293 cells (constructed by GeneScience, on the surface of which VEGFA's receptor KDR is expressed; when VEGFA binds to the KDR on the surface of HEK293 cells, the downstream signaling pathway is activated, and the binding of NFAT to NFAT cis-element leads to Luc2P expression to generate fluorescence) were seeded in a 96-well plate at 30,000/50 μl/well. Each antibody was diluted in a 3-fold gradient with a total of 10 concentrations and a highest final concentration of 1 μM (stock concentration of 4 Mm, 25 μl/well); and the final concentration of VEGFA protein was 50 ng/ml (stock concentration of 200 ng/ml, 25 μl/well). Cells were lysed after 4 h incubation in the incubator, and the reporter gene was detected. The results are shown in FIG. 9, which demonstrates that the blocking activity of the preferred bispecific molecules on the VEGFA signal is comparable to that of the control molecule Eylea.

### Example 6 Determination of T Cells Activation Activity of Preferred Antibodies

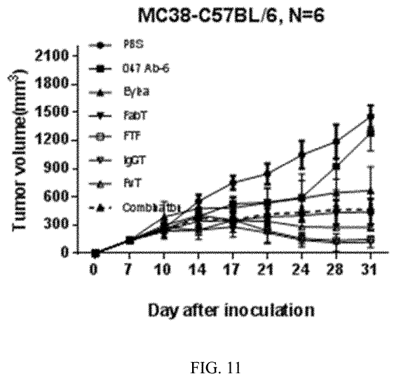

CHO-PDL1-CD3L cells (constructed by GeneScience, on the surface of which PDL1 and CD3L are expressed) were seeded in a 96-well plate at 40,000/well and placed in an incubator overnight to adhere. 047 Ab-6 control sample and four samples of bispecific antibody fusion proteins (FabT, FTF, IgGT and FvT) were subjected to test. The samples were diluted in a 3-fold gradient with a total of 10 concentrations and an initial concentration of 687.5 nM. After addition of diluted antibody, Jurkat-PD1-NFAT cells (constructed by GeneScience, on the surface of which PD1 is expressed; when the CD3L on the surface of CHO cells binds to Jurkat cells, the signal pathway in CHO cells is activated to therefore generate fluorescence, and when the PDL1 on the surface of CHO cells binds to the PD1 on the surface of Jurkat cells, NFAT signal pathway will be blocked and unable to generate fluorescence) were seeded in a culture plate at 100,000/well. The cells and antibodies were gently mixed and incubated for 6 h, and then Bio-glo was added for detection. The results are shown in FIG. 10, which demonstrates that the cell activation activities of FabT, FTF, IgGT and FvT were slightly lower than that of the control antibody 047 Ab-6.

### Example 7 Drug Efficacy Determination of Preferred Antibodies in Animals

Model: C57BL/6 mice subcutaneously inoculated with MC38 cells (colorectal cancer cells) at 2×10⁵ cells/mouse

Administration: When the subcutaneous tumor volume reached about 100-150 mm³, mice were randomly divided into groups with 6 mice in each group, and administered intraperitoneally, twice a week for a total of 3 weeks: 047 Ab-6, 3 mg/kg; Eylea, 2 mg/kg; combination administration, 047 Ab-6+Eylea, 3 mg/kg+2 mg/kg, administered in the morning and evening; FabT, 2 mg/kg; FTF, 3.8 mg/kg; IgGT, 3.8 mg/kg; and FvT, 3.8 mg/kg.

The results are shown in FIG. 10 and Table 8. The tumor inhibition rate of the control antibodies 047 Ab-6 and Eylea was 12% and 54%, respectively, and the tumor inhibition rate of the combination of 047 Ab-6 and Eylea reached 68%. The tumor inhibition rate of the bispecific antibody fusion proteins of the present invention was higher than that of combination of 047 Ab-6 and Eylea, and the tumor inhibition rates of FTF and IgGT reached 90% and 92%, respectively.

TABLE 8
Tumor inhibition rate of candidate bispecific molecules

| Drug | TGI (%) |
|---|---|
| 047 Ab-6 | 12 |
| Eylea | 54 |
| 047 Ab-6 + Eylea | 68 |
| FabT | 70 |
| FTF | 90 |
| IgGT | 92 |
| FvT | 81 |

The antibody fusion protein, preparation method thereof and application thereof provided by the present disclosure are described in detail above. Specific examples are given herein to illustrate the principle and embodiments of the present disclosure, and the illustration of these examples is only intended to facilitate understanding of the methods of the present disclosure and core concept thereof. It should be noted that, several improvements and modifications may be made by those skilled in the art to the present disclosure without departing from the principle of the present disclosure, and these improvements and modifications also fall within the protection scope of the claims thereof.

1-2. (canceled)

3. An antibody fusion protein, comprising
a1). light chain variable region and light chain constant region of the antibody that specifically binds to the first antigen, the flexible peptide and the fusion protein that specifically binds to the second antigen, represented as VL-CL-linker-Trap, and
b1). heavy chain variable region, heavy chain constant region 1 and partial hinge region of the antibody that specifically binds to the first antigen, the flexible peptide, and the fusion protein that specifically binds to the second antigen, represented as VH-CH1-Partial hinge-linker-Trap;
or comprising
a2). light chain of the antibody that specifically binds to the first antigen, and
b2). heavy chain variable region and heavy chain constant region 1 of the antibody that specifically binds to the first antigen, the flexible peptide, the fusion protein that specifically binds to the second antigen, heavy chain constant region 2 and heavy chain constant region 3, represented as VH-CH1-linker-Trap-CH2-CH3;
or comprising
a3). light chain of the antibody that specifically binds to the first antigen, and
b3). heavy chain variable region, heavy chain constant region 1, heavy chain constant region 2 and heavy chain constant region 3 of the antibody that specifically binds to the first antigen, the flexible peptide, and the fusion protein that specifically binds to the second antigen, represented as VH-CH1-CH2-CH3-linker-Trap;
or comprising
a4). light chain variable region of the antibody that specifically binds to the first antigen, the flexible peptide, the fusion protein that specifically binds to the second antigen, heavy chain constant region 2 and heavy chain constant region 3, represented as VL-linker-Trap-CH2-CH3, and
b4). heavy chain variable region of the antibody that specifically binds to the first antigen, the flexible peptide, the fusion protein that specifically binds to the second antigen, heavy chain constant region 2 and heavy chain constant region 3, represented as VH-linker-Trap-CH2-CH3; and
wherein the antibody fusion protein specifically binds to hPD-L1 and hVEGF-A; and the first antigen is hPD-L1 and the second antigen is hVEGF-A.

4. The antibody fusion protein of claim 3, wherein the flexible peptide comprises a sequence of (G4S)ₙ, wherein n is an integer greater than 0; preferably an integer of 1-10.

5. The antibody fusion protein of claim 3, wherein the light chain constant region and the heavy chain constant region 1 form a heterodimer, and the terminal cysteine residue in the light chain constant region and the cysteine residue in the hinge region of heavy chain form a disulfide bond.

6. The antibody fusion protein of claim 3, wherein the cysteine residues in the hinge regions of heavy chains form a disulfide bond.

7. The antibody fusion protein of claim 3, wherein the domain of the heavy chain constant region 3 of first heavy chain and the domain of the heavy chain constant region 3 of second heavy chain are modified to a structure that facilitates the formation of the antibody fusion protein.

8. The antibody fusion protein of claim 7, wherein the modification comprises
c) modification to the domain of the heavy chain constant region 3 of the first heavy chain: in the interface between the domain of the heavy chain constant region 3 of the first heavy chain and the domain of the heavy chain constant region 3 of the second heavy chain of bivalent bispecific antibody, an amino acid residue in the domain of the heavy chain constant region 3 of the first heavy chain is replaced with an amino acid residue with a volume larger than the original amino acid residue to form a knob in the domain of the heavy chain constant region 3 of the first heavy chain, wherein the knob is capable of inserting into a hole of the domain of the heavy chain constant region 3 of the second heavy chain, and
d) modification to the domain of the heavy chain constant region 3 of the second heavy chain: in the interface between the domain of the heavy chain constant region 3 of the second heavy chain and the domain of the heavy chain constant region 3 of the first heavy chain of bivalent bispecific antibody, an amino acid residue in the domain of the heavy chain constant region 3 of the second heavy chain is replaced with an amino acid residue with a volume smaller than the original amino acid residue to form a hole in the domain of the heavy chain constant region 3 of the second heavy chain, wherein the hole is capable of holding the knob of the domain of the heavy chain constant region 3 of the first heavy chain.

9. The antibody fusion protein of claim 8, wherein in the heavy chain,
the amino acid residue with a volume larger than the original amino acid residue is selected from the group consisting of arginine, phenylalanine, tyrosine, and tryptophan; and
the amino acid residue with a volume smaller than the original amino acid residue is selected from the group consisting of alanine, serine, threonine, and valine.

10. The antibody fusion protein of claim 3, comprising
(IV) the heavy chain with an amino acid sequence as shown in SEQ ID NO: 4 or SEQ ID NO: 5 or SEQ ID NO: 6 or SEQ ID NO: 7, and (V) the light chain with an amino acid sequence as shown in SEQ ID NO: 8 or SEQ ID NO: 9 or SEQ ID NO: 10;
or
(VI) an amino acid sequence derived from the amino acid sequence described in (IV) or (V) by substitution, deletion or addition of one or more amino acids, and functionally identical or similar to the amino acid sequence described in (IV) or (V);
or
(VII) an amino acid sequence with more than 90% homology with the sequence described in (IV) or (V);
or
(VIII) an amino acid sequence that has the same functional fragment or functional variant as the sequence described in (IV) or (V);
wherein the antibody fusion protein specifically binds to hPD-L1 and hVEGF-A; and
the first antigen is hPD-L1 and the second antigen is hVEGF-A.

11. The antibody fusion protein of claim 10, wherein the one or more amino acids is 2-10 amino acids.

12. The antibody fusion protein of claim 3, comprising
(IX) a heavy chain with an amino acid sequence of SEQ ID NO: 4, and a light chain with an amino acid sequence of SEQ ID NO: 8; or
(X) a heavy chain with an amino acid sequence of SEQ ID NO: 5, and a light chain with an amino acid sequence of SEQ ID NO: 9; or
(XI) a heavy chain with an amino acid sequence of SEQ ID NO: 6, and a light chain with an amino acid sequence of SEQ ID NO: 9; or
(XII) a heavy chain with an amino acid sequence of SEQ ID NO: 7, and a light chain with an amino acid sequence of SEQ ID NO: 10.

13. A nucleic acid molecule encoding the antibody fusion protein of claim 3, comprising
(XIII) a nucleic acid encoding the heavy chain variable region as shown in SEQ ID NO: 4 or SEQ ID NO: 5 or SEQ ID NO: 6 or SEQ ID NO: 7, and a nucleic acid encoding the light chain variable region as shown in SEQ ID NO: 8 or SEQ ID NO: 9 or SEQ ID NO: 10;
or
(XIV) a nucleic acid having complementary sequence of the heavy chain variable region as shown in SEQ ID NO: 4 or SEQ ID NO: 5 or SEQ ID NO: 6 or SEQ ID NO: 7, and a nucleic acid having complementary sequence of the light chain variable region as shown in SEQ ID NO: 8 or SEQ ID NO: 9 or SEQ ID NO: 10;
or
(XV) a nucleotide sequence encoding the same protein as the nucleotide sequence described in (XIII) or (XIV), but different from the nucleotide sequence described in (XIII) or (XIV) due to the degeneracy of genetic code;
or
(XVI) a sequence with more than 90% homology with the sequence described in (XIII) or (XIV) or (XV).

14. The nucleic acid molecule of claim 13, comprising a nucleotide sequence derived from the nucleotide sequence described in (XIII) or (XIV) or (XV) or (XVI) by substitution, deletion or addition of one or more nucleotide, and functionally identical or similar to the nucleotide sequence described in (XIII) or (XIV) or (XV) or (XVI), wherein the one or more amino acids is 2-10 amino acids.

15. An expression vector, comprising the nucleic acid molecule of claim 13, and a cell transformed with the expression vector.

16. A complex, comprising the antibody fusion protein of claim 3 covalently linked to an isotope, an immunotoxin and/or a chemical drug.

17. A conjugate, formed by coupling the antibody fusion protein of claim 3 with a solid medium or a semi-solid medium.

18. (canceled)

19. A pharmaceutical composition, comprising the antibody fusion protein of claim 3.

20. A kit, comprising the antibody fusion protein of claim 3.

21. A method for treating a disease comprising administering the antibody fusion protein of claim 3 to a subject in need thereof, wherein the disease is selected from the group consisting of breast cancer, lung cancer, gastric cancer, intestinal cancer, esophageal cancer, ovarian cancer, cervical cancer, kidney cancer, bladder cancer, pancreatic cancer, non-Hodgkin's lymphoma, chronic lymphoma leukemia, multiple myeloma, acute myeloid leukemia, acute lymphoma leukemia, glioma, melanoma, diabetic macular edema, and wet macular degeneration.

22. A method for producing the antibody fusion protein of claim 3, comprising transforming a host cell with the expression vector comprising the nucleic acid molecule encoding the antibody fusion protein, culturing the host cell under conditions that allow the synthesis of the antibody fusion protein, and recovering the antibody fusion protein from the culture.

23. The antibody fusion protein of claim 3, wherein the amino acid sequence of the fusion protein that specifically binds to the second antigen is represented by SEQ ID NO: 1.

24. The antibody fusion protein of claim 3, wherein the amino acid sequence of the light chain variable region of the antibody that specifically binds to the first antigen is represented by SEQ ID NO: 2; the amino acid sequence of the heavy chain variable region of the antibody that specifically binds to the first antigen is represented by SEQ ID NO: 3.
